(19) United States
(12) Patent Application Publication
(10) Pub. No.: US 2019/0144808 A1
(43) Pub. Date: May 16, 2019

(54) WELL INCLUDING A PIT

(71) Applicant: THE RESEARCH FOUNDATION FOR THE STATE UNIVERSITY OF NEW YORK, Albany, NY (US)

(72) Inventors: Jian CAO, S. Setauket, NY (US); Vincent ALFORD, Coral Springs, FL (US); Yizhi MENG, Centereach, NY (US)

(73) Assignee: THE RESEARCH FOUNDATION FOR THE STATE UNIVERSITY OF NEW YORK, Albany, NY (US)

(21) Appl. No.: 16/306,947

(22) PCT Filed: Jun. 6, 2017
(86) PCT No.: PCT/US2017/036116

§ 371 Date: Dec. 4, 2018

Related U.S. Application Data
(60) Provisional application No. 62/346,141, filed on Jun. 6, 2016.

(51) Int. Cl. C12M 1/32; C12M 1/00; C12M 1/12; C12M 1/34

(52) U.S. Cl. CPC C12M 23/12; C12M 41/46; C12M 25/14; C12M 23/34

(57) ABSTRACT

The present disclosure is directed to a plate. The plate can include one or more wells, wherein each of the one or more wells comprises a bottom surface, a pit formed in the bottom surface, and a pit barrier formed around a periphery of the pit.

### CROSS REFERENCE TO RELATED APPLICATIONS

The present application claims benefit of U.S. Provisional Application No. 62/346,141, filed Jun. 6, 2016, the entire contents of which are incorporated herein by reference.

### STATEMENT REGARDING FEDERALLY SPONSORED RESEARCH OR DEVELOPMENT

This invention was made with government support under grant number CA166936 awarded by the National Institutes of Health. The government has certain rights in the invention.

### BACKGROUND OF THE DISCLOSURE

A life-threatening aspect of cancer is metastasis. Steps in this disseminated process include cell migration and invasion of tumor cells into the vasculature and surrounding matrices. Metastatic cancers that spread to other tissues distant from the original tumor site are quite serious.

Evidence suggests that at the time of diagnosis, a high proportion of patients already have micrometastasis. Presently available diagnostic markers for cancer often fail to detect cancerous cells early enough for therapeutic intervention to prevent metastasis.

However, due to limitations in drug discovery approaches, progress in targeting cancer invasiveness has plateaued. Instead, most anti-cancer compounds developed are cytotoxic and are associated with severe side effects due to the inability to selectively target cancer cells. Additionally, these drugs have little-to-no impact on reducing metastasis, which contributes to 90% of all human cancer-related mortalities. Despite considerable investment in cytotoxic drug discovery, their limited use against solid tumors coupled with their failure to prevent metastasis has reinforced the unmet need of targeting cancer invasion. Since targeting and preventing cancer metastasis remains an obstacle in effective patient care, a novel anti-cancer drug approach which targets cancer invasion is needed to solve this problem currently unaddressed by modern medicine.

Effective targeting of cancer cell invasion requires an amenable technology to mimic in vivo conditions. Mounting evidence has demonstrated that cancer cells cultured in 3D matrices more closely mimic in vivo conditions when compared to traditional 2D cell culture methods. Of the 3D high-throughput screening assays available, nearly all are focused on identifying compounds that inhibit tumor growth (increase in cell mass) and proliferation (increase in cell number).

Embodiments of the present disclosure provide methods that address the above and other issues.

### SUMMARY OF THE DISCLOSURE

The present disclosure is directed to a plate useful for 3D high-throughput screening cell invasion assays. The plate can include one or more wells, wherein each of the one or more wells comprises a bottom surface, a pit formed in the bottom surface, and a pit barrier formed around a periphery of the pit.

### BRIEF DESCRIPTION OF THE DRAWINGS

The present disclosure will be better understood by reference to the following drawings of which:

FIGS. 1a-1e are graphical illustrations of well, pit and the introduction of the various components to the well.

FIG. 2 is a magnified, cross sectional view of a portion of one embodiment of the pit and the pit barrier.

FIG. 3 is a magnified, cross sectional view of a portion of one embodiment of the pit and the pit barrier.

FIG. 4 is a magnified, cross sectional view of a portion of one embodiment of the pit and the pit barrier.

FIG. 5 is a magnified, cross sectional view of a portion of one embodiment of the pit and the pit barrier.

FIG. 6 is a magnified, cross sectional view of one embodiment of the pit and the pit barrier with a cell-matrix mixture added to the pit.

FIGS. 7a-7e are photographs of cells that have exited the cell-matrix mixture in the pit.

FIGS. 8a-8c are cross sectional views of embodiments of pits and pit barriers.

FIGS. 9a and 9b are fluorescence microscopy images.

FIGS. 10a-10d are fluorescence microscopy images.

FIGS. 11a-11d are fluorescence microscopy images.

### DETAILED DESCRIPTION OF THE DISCLOSURE

In the discussion and claims herein, the term “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

As used herein terms such as “a”, “an” and “the” are not intended to refer to only a singular entity, but include the general class of which a specific example may be used for illustration.

As used herein, terms defined in the singular are intended to include those terms defined in the plural and vice versa.

As used herein, the terms “inhibit”, “suppress” or “silence” refer to the act of diminishing, alleviating, preventing, reducing or eliminating. For example, a compound that inhibits cancer may kill all cancerous cells or prevent, arrest or slow further cancerous cell growth. These terms find use in both in vitro as well as in vivo systems, as for example the inhibiting the growth or proliferation of cancer cells. It is not necessary that there be complete inhibition, suppression or silencing, for the present application it is sufficient for there to be some inhibition, suppression or silencing.

As used herein, the terms “cancer cell” and “tumor cell” refer to a, cell undergoing early, intermediate or advanced stages of multi-step neoplastic progression as previously described (Pitot et al., Fundamentals of Oncology, 15-28 (1978)), herein incorporated by reference. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. A cell in the early stages of malignant progression is referred to as a “hyperplastic cell” and is characterized by dividing without control and/or at a, greater rate than a normal cell of the same cell type in the same tissue. Proliferation may be slow or rapid but continues unabated. A cell in the intermediate stages of neoplastic progression is referred to as a “dysplastic cell”. A dysplastic cell resembles an immature epithelial cell, is generally spatially disorganized within the tissue and loses its specialized structures and functions. During the intermediate stages of neoplastic progressions an increasing percentage of the epithelium becomes composed of dysplastic cells, “Hyperplastic” and “dysplastic” cells are referred to as “pre-neoplastic” cells. In the advanced stages of neoplastic progression a dysplastic cell become a “neoplastic” cell. Neoplastic cells are typically invasive i.e., they either invade adjacent tissues, or are shed from the primary site and circulate through the blood and lymph to other locations in the body where they initiate secondary cancers. The term “cancer” or “neoplasia” refers to a plurality of cancer cells. “Aggressive cancer cells” are cancer cells that are capable of metastasizing.

As used herein, the term “carcinoma” refers to a malignant new growth made up of epithelial cells tending to infiltrate the surrounding tissues and give rise to metastases.

As used herein, the terms “metastasis”, “metastatic” and “metastasize” refer to the transfer of abnormal cells from one primary site, organ or location to another not directly connected with it to form new foci of disease due the transfer of cells, as in malignant tumors. The capacity to metastasize is a characteristic of all malignant tumors.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes whole numbers of 5, 6, 7, 8, 9, and 10, and fractional numbers 5.1, 5.2, 5.3, 5.4, 5.5, 5.6, 5.7, 5.8, 5.9, etc.

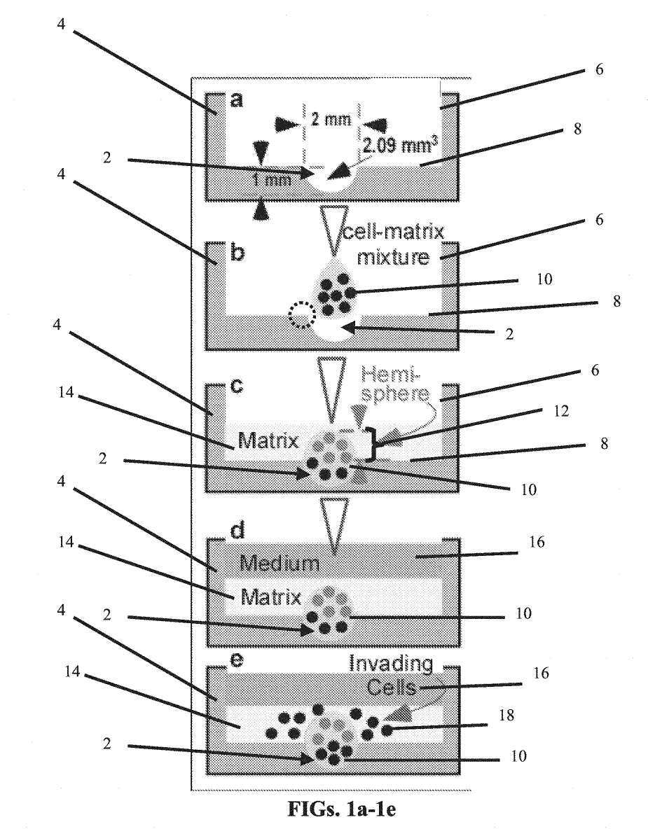

As used herein, a “well” is a cavity, indentation, space, dent, crater, depression, hollow, recess or impression that is formed in the surface of a structure. The cross section of a well that is used for cell culture may be any shape, including, but not limited to, cross sections with curved lines (e.g., with a, hemispheric and/or semicircular well bottom, straight line (e.g., flat well bottoms), converging straight lines (e.g., “V” shaped well bottom) and a combination of a flat well bottom and “V” shaped side walls. Thus, cross sectional shape in plan view include, square, round, hexagonal, other geometric or non-geometric shapes, and combinations (intra-well and inter-well) thereof. Cross sectional shapes in vertical views include shear vertical or chamfered walls, wells with flat or round bottoms, conical walls with flat or round bottoms, and curved vertical walls with flat or round bottoms, and combinations thereof. For example, a well is depicted in FIG. 1a as reference number 4.

The wells of the disclosure can be included in any other suitable structure or plate. For example, the wells of the disclosure can be included in any suitable single or multi-well plate and tube array, in, e.g. 1-, 2-, 4-, 6-, 12-, 24-, 48-, 96-, 384-, and 1536-well designs. Multi-well plates can be used for any suitable purpose, one example of which being use as a tissue culture plate. Multi-well plates and tube arrays can be made of any suitable plastic material, including polyolefins such as polystyrene, polypropylene, siloxanes such as polydimethylsiloxane (PDMS) and others in virgin state or mixed with other materials in order to provide clear, white and/or black shades.

As used herein, the term “pit” refers to a depression, indentation, secondary well, concave portion, notch, dent or dimple formed in a bottom surface of a well, such that the lowest portion of the pit is below the bottom surface of the well. The cross section of a pit can be any shape, including, but not limited to, cross sections with curved lines (e.g., with a hemispheric and/or semicircular well bottom, straight line (e.g., flat well bottoms), converging straight lines (e.g., “V” shaped well bottom). Thus, cross sectional shape in plan view include, square, round, hexagonal, other geometric or non-geometric shapes, and combinations (intra-well and inter-well) thereof. Cross sectional shapes in vertical views include shear vertical or chamfered walls, wells with flat or round bottoms, conical walls with flat or round bottoms, and curved vertical walls with flat or round bottoms, and combinations thereof. For example, a pit is depicted in FIG. 1a as reference number 2.

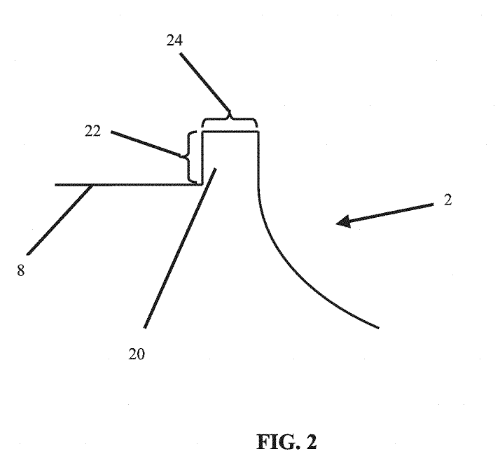

As used herein, the term “pit barrier” refers to a flange, post, protrusion, projection, lip or extension that extends vertically above the pit and the bottom surface of the well and that extends around the circumference or periphery of the pit. The pit barrier can have any suitable cross sectional area, including cross sections with curved lines and straight lines, forming cross sections of various shapes including but not limited to substantially rectangular shapes and substantially triangular shapes. For example, a pit barrier is depicted in FIG. 2 as reference number 20.

One view of a well of the present disclosure is shown in FIGS. 1a-1e. The dimensions shown in this well are for discussion purposes only and are not intended to restrict the disclosure in any way. Further, the elements shown in FIGS. 1a-1e are not to scale. This well also does not include a pit barrier, as described below.

As shown in FIG. 1a, a substantially hemispherical pit 2 is contained within a well 4, the well having substantially vertical walls 6 around its exterior. The substantially hemispherical pit 2 can be formed anywhere on a lower surface 8 of the well 4, in this figure the substantially hemispherical pit 2 is formed around the center of the well 4. As can be seen from FIG. 1a, the bottom surface 8 is continuous with the vertical walls 6 of the well 4. As can also be seen the pit 2 is continuous with the bottom surface 8. The hemispherical pit 2 can be any suitable radius, such as from 0.1 mm, 0.5 mm, 1 mm, 1.5 mm, 2 mm or more.

In one embodiment, the disclosure provides a multi-well platform comprising a plurality of wells, one or more of which include a pit barrier, as discussed above. Note that each of the wells does not need to include the pit. The multi-well platforms of the present disclosure comprise a frame that can be made of any material, such as polymers (e.g., polystyrene, cycloolefins, etc.), glass, quartz, etc. The frame can be of any thickness, such as from 0.5, 1, 2, 3, or 5 millimeters to 2, 3, 5, 10 or 20 millimeters, or more.

The frame can be of any shape, and defines the footprint of the multi-well platform (e.g., square, rectangular, circular, oblong, triangular, kidney, or other geometric or non-geometric shape). The footprint can have a shape that is substantially similar to the footprint of existing multi-well platforms, such as a 96-well microtiter plate, whose footprint is about 85.5 mm in width by about 127.75 mm in length or other sizes.

Wells can be arranged in two-dimensional linear arrays on the multi-well platform. However, the wells can be provided in any type of array, such as geometric or non-geometric arrays. Some examples of the number of wells of the array are 1, 2, 4, 8, 12, 16, 24, 96, 384, 864, 1536, 3456, and 9600, including all numbers in between.

Well volumes can vary depending on well depth and cross sectional area. For example, well volumes can range from about 0.5, about 1, about 5, about 10, about 25, about 50, about 75, about 100 or about 200 μL to about 5, about 15, about 40, about 80, about 100, about 200, about 500, or about 1,000 μL.

In FIG. 1b, a cell-matrix mixture 10 is added to pit 2 by any suitable mechanism, in this figure the cell-matrix mixture 10 is added by pipette. The cell-matrix mixture 10 is a combination of a number of cells, e.g. cancer cells, and a suitable liquid carrier, (such as a gel) within which the cells are enclosed or embedded. “Gel” refers to a semi-solid colloid in a more solid form than a liquid, and a more liquid form than a solid. Examples of matrices are disclosed in (Kim et al. (2004) Breast Cancer Research and Treatment 85:281-291; U.S. Patent Application Publication No. US 2006/0003311 to Fulde et al., and Debnath et al. 92005) Nature Reviews 5:675-688). Examples of matrices include natural scaffolds and synthetic scaffolds. Natural scaffolds include collagen (such as pre-engineered collagen scaffolds), matrigel (e.g., from BD) Biosciences), alginate, agarose, hyaluronic acid, and proteoglycan. Synthetic scaffolds include Skelite™, Poly (2-hydroxyethyl methacrylate) (polyHEMA), polyglycolic acid (PGA), polylactic acid (PLA), and mixtures of PGA and PLA.

The cells of the cell-matrix mixture 10 can be cells where their migration or invasion capabilities are of interest. The concentration of cells in the cell-matrix mixture 10 can be any suitable concentration, such as about 1×10³ to about 1×10¹⁰ cells/mL, or about 1×10⁴ cells/mL, or about 1×10⁹ cells/mL, or about 1×10⁵ cells/mL, or about 1×10⁸ cells/mL, or about 1×10⁶ cells/mL, or about 1×10⁷ cells/mL, or about 2×10⁷ cells/mL.

In FIG. 1c, the cell-matrix mixture 10 is within pit 2, forming a, hemispherical portion 12 above the lower surface 8 of the well 4. A matrix 14 is added to well 4 to substantially cover cell-matrix mixture 10. Matrix 14 can be any suitable medium, such as for example a collagen material. Matrix 14 can also include various components, including inhibitors or vehicle controllers that could potentially affect the migration or invasion of the cells of the cell-matrix mixture 10.

Matrix 14 can include various components such as pharmacologic agents (drugs, nutraceuticals, small molecules etc.). In some embodiments, the cells or cancer cells of cell-matrix mixture 10 have been previously transfected with green fluorescent protein (GFP) cDNA to permit visualization of cancer invasion of the matrix 14 and to permit automation of the technique.

Invading cancer cells leaving the perimeter of the cell-matrix mixture 10 can be visualized extending into the surrounding collagen matrix after about 18 hours of incubation. Both qualitative and quantitative analyses can be performed microscopically. The invasive ability of cancer cells (cells outside the cell-matrix mixture 10), as well as the effect of pharmacologic agents on the cancer cell invasion, can be determined microscopically. The effect of pharmacologic agents on invasion of cancer cells is determined by counting invading cells in the presence versus the absence of pharmacologic agents.

Inhibition of in vitro cancer invasion identifies drugs that have the potential to be useful in cancer treatment. This assay will be readily adaptable for high throughput screening of libraries of compounds that may be useful for cancer treatment. This assay is also useful for identifying drugs capable of interfering with invasion of other cell types such as endothelial cells, nerve cells, etc. In addition, this assay can provide a rapid, convenient, and reliable approach to evaluate cancer invasion-related genes or anti-metastasis-related genes.

In FIG. 1d, a medium 16 is added to well 4, substantially covering matrix 14. Medium 16 can be any suitable cell culture medium.

After a period of time some of the cells of the cell-matrix mix mixture 10 migrate or invade into the matrix 14, as shown in FIG. 1e. These invasion cells 18 can be counted using any suitable equipment and technique to determine the mobility of the cells of the matrix mixture 10 and the inhibition of mobility of the matrix 14 and what various components are included in matrix 14.

A more detailed discussion of the well 4, pit 2 and lower surface 8 is provided below. FIG. 2 is a magnified view of the dashed circle of FIG. 1b.

FIG. 2 includes a pit barrier 20, which extends a distance above lower surface 8 and extends the edge of the pit 2. FIG. 2 is a cross sectional view, but the pit barrier 20 extends around a periphery of pit 2. A height 22 of pit barrier 20 can be any suitable distance from lower surface 8, such as, in the range of about 0.001 mm to about 1 mm or more. In other embodiments, height 22 can be any suitable height in the range of about 0.01 mm to about 1 mm, or about 0.02 mm to about 1 mm, or about 0.1 mm to about 1 mm, and all distances in between these ranges.

As can be seen from FIG. 2, the pit 2, the pit barrier 20 and the bottom surface 8 are continuous with each other, forming a continuous surface.

A width 24 of the upper portion of pit barrier 20 can be any suitable distance, such as, in the range of about 0.001 mm to about 1 mm or more. In other embodiments, height 22 can be any suitable height in the range of about 0.01 mm to about 1 mm, or about 0.02 mm to about 1 mm, or about 0.1 mm to about 1 mm, and all distances in between these ranges. In FIG. 2 for example, width 24 of the upper portion can be about 0.01 mm.

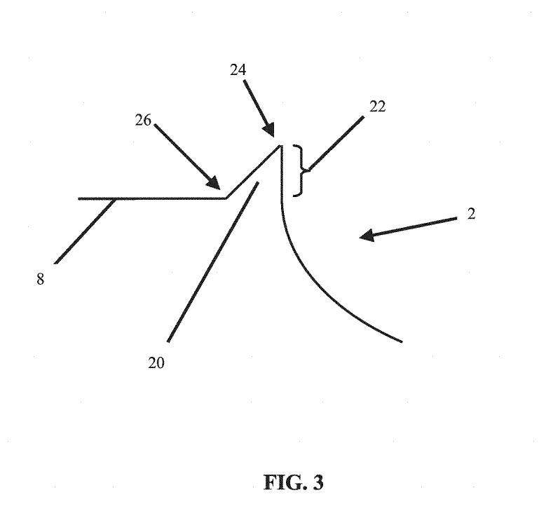

The shape of pit barrier 20 can be modified, such as the width 24 can be reduced to substantially form a point, for example width 24 can be about 0.01 mm, as shown in FIG. 3. A transition point 26 between pit barrier 20 and lower surface 8 is shown at an angle, but, in other embodiments, the transition point 26 could be curved or any be other suitable cross sectional shape.

As shown in FIG. 3 the angle formed between the edge of the pit 2 and the height 22 of the pit barrier is shown as substantially the same angle (or about 180°), but in other embodiments this angle can be modified from about 120° or less to about 240° or more, and all angles in between. In FIG. 3 for example, width 24 of the upper portion can be about 0.01 mm.

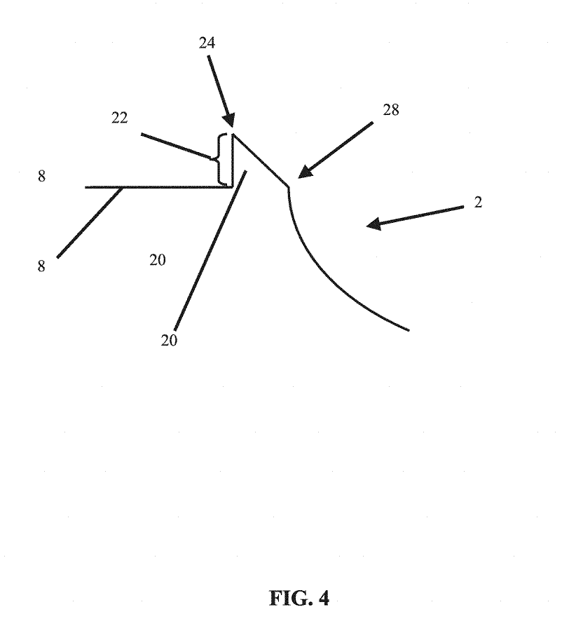

Another embodiment where width 24 is reduced substantially to form a point is shown in FIG. 4. In FIG. 4 a transition point 28 between the edge of pit 2 and the pit barrier 20, the transition point 28 is shown at an angle, but, in other embodiments, the transition point 28 could be curved or be any other suitable cross sectional shape.

As shown in FIG. 4 the angle formed between the pit barrier 20 and the lower surface 8 is about 90°, but in other embodiments this angle can be modified from about 30° or less to about 150° or more, and all angles in between. In FIG. 4 for example, width 24 of the upper portion can be about 0.01 mm.

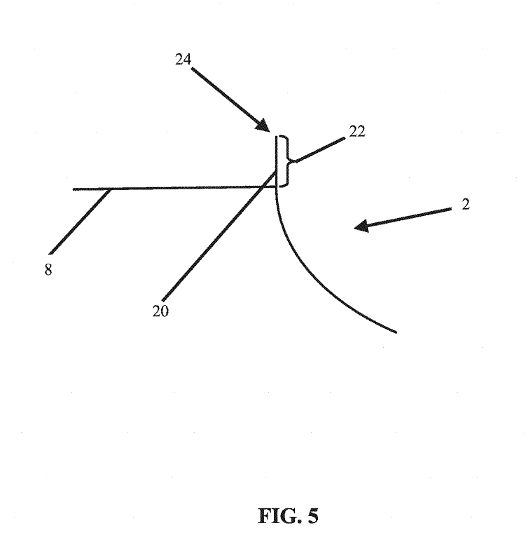

Another embodiment is shown in FIG. 5, where width 24 is reduced substantially to form a point along the height 24 of pit barrier 20. As shown in FIG. 5 the angle formed between the edge of the pit 2 and the height 22 of the pit barrier is shown as substantially the same angle (or about 180°), but in other embodiments this angle can be modified from about 120° or less to about 240° or more, and all angles in between. In FIG. 52 for example, width 24 of the upper portion can be about 0.01 mm.

In each of the discussed figures the face of pit barrier 20 facing away from pit 2 is shown as being straight, but, in other embodiments, this portion could be curved or any other suitable cross sectional shape. In each of the discussed figures the face of pit barrier 20 facing towards pit 2 is shown as being straight, but, in other embodiments, this portion could be curved or any other suitable cross sectional shape.

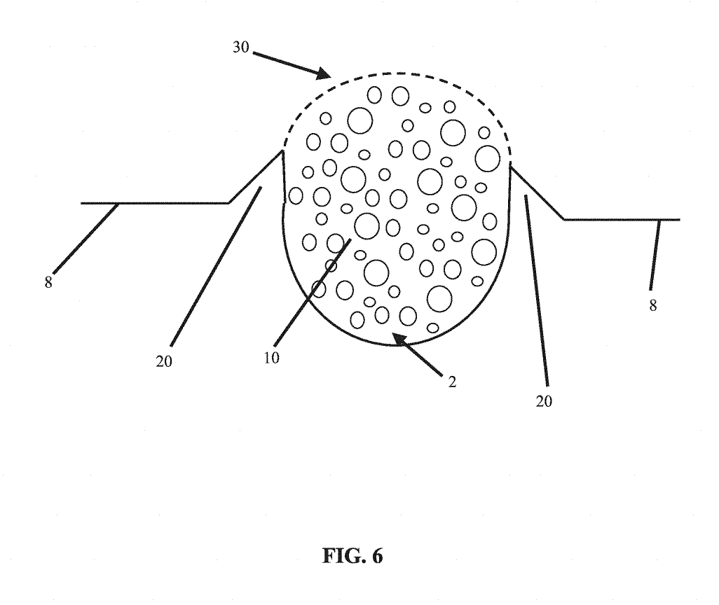

One embodiment of a pit 2 is shown in FIG. 6, with cell-matrix mixture 10 being already added to pit 2. As can be seen a portion of cell-matrix mixture 10 extends vertically above pit barrier 20, forming a meniscus 30, as indicated by the dashed line. Although the height of meniscus 30 extending vertically above pit barrier 20 will change based on the specified size of each, in one embodiment the meniscus 30 extends about 200 μm above pit barrier 20, but in other embodiments this distance can change by up to 100%, 200%, 300%, or more.

The inclusion of pit barrier 20 allows for a meniscus that protrudes further from lower surface 8 as compared to any meniscus formed without a pit barrier. This meniscus 30 also can allow for more migration of cells from the cell-matrix mixture 10, and can also make imaging of those migrated cells easier and/or more accurate.

Further, due to at least an increased surface tension of meniscus 30 created due to pit barrier 20, the cell-matrix mixture 10 is less likely to spill out of pit 2 onto lower surface 8 during operation.

In another embodiment of the disclosure, one or more wells of an assay plate can have a cell-matrix mixture added to them, into the respective pits of the one or more wells. In this embodiment each of the pits of the one or more wells includes a pit barrier around its periphery. After the cell-matrix mixture is added, a matrix 14 can be placed in the well and substantially cover the cell-matrix mixture. At this point, and before incubation to effect migration of cells out of the cell-matrix mixture, the assay plate can be preserved (such as by reducing its temperature) and stored for later use by the party that added the cell-matrix mixture to the well, or another third party.

The methods, apparatus and compositions of the present disclosure will be better understood by reference to the following Examples, which are provided as exemplary of the disclosure and not by way of limitation.

### Example 1

The following methodology permits the detection of cancer cell invasion into a collagen matrix and the inhibitory activity of anti-cancer drugs. The disclosed assay can be useful in identifying pharmacologic agents (drugs) that interfere with cancer progression.

Preliminary data now demonstrate an assay for quantitative evaluation of cancer cell migration/invasion in a 3D matrix that is simple, precise and easy to replicate. This assay allows simultaneous observation of cell migration, cell invasion and cell death using image-based analysis, and may be converted into a fully automated high-throughput 3D invasion assay to accelerate anti-cancer drug discovery for treatment/prevention of metastasis.

In this example a mixture of cells (2×10⁷ cells/mL, final concentration) and type I collagen (1.5 mg/mL, final concentration) were loaded into the pits of a manufactured 96-well plate, one example of which is discussed in reference to FIGS. 1a-1e above.

Based on the size of the pit, the volume of the cell-matrix mixture added to create a sphere in each pit is 4.18 mm³ (μL) [4/3πr³, r=1 mm]. The plates were transferred to a 37° C. incubator for 10 min to allow gelation followed by the addition of 70 μL of type I collagen (1.5 mg/ml) to cover each cell-matrix hemisphere. After a, 20 min incubation at 37° C., the assembled cell invasion complex was then covered with 70 μL of cell culture medium with added inhibitors or vehicle control and incubated for 24 hours at 37° C.

To determine the number of cells that invaded the type I collagen matrix, the cells were incubated with propidium iodide (PI) (to identify dead cells due to cytotoxic effects), and 4′,6-diamidino-2-phenylindole (DAPI) (to identify cells for accuracy of cell counts) followed by fixation with 4% paraformaldehyde. The first well in the plate dimethyl sulfoxide (DMSO vehicle control) was manually focused and set as a standard setting for automated scanning. Four images were acquired to cover the entire tumor sphere located in each well at one focal plane.

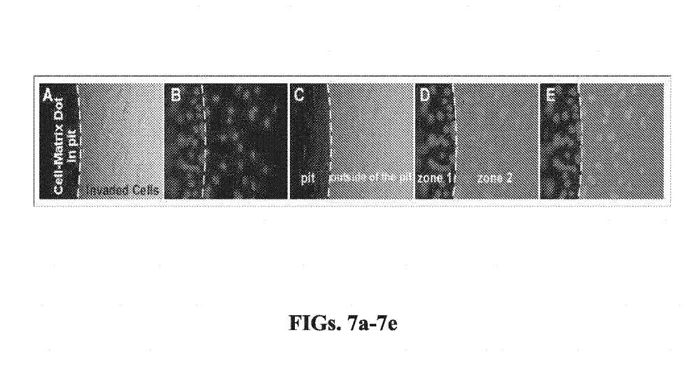

The remaining wells on the plate were scanned under control of the NIS-Elements imaging software. To quantify the number of cells that invade into the surrounding matrix, both phase contrast and fluorescent imaginings of DAPI and PI were acquired. A 10× objective lens, a Nikon charge-coupled device (CCD) camera, and a, motorized stage scanned one plate in approximately 20 minutes [6 min/plate×3 (phase & DAPI/fluorescence & PI/fluorescence)+shutter switch time] at a single focal plane. The images were be stored and the number of invaded cells were quantified by creating a binary zone that distinguishes the initial cell-matrix sphere within the pits from the invading zone as illustrated in FIGS. 7a-7e.

In FIGS. 7a-7e human fibrosarcoma HT1080 cells were evaluated for their invasive ability using the 3-D invasion assay. After an 18 hour incubation, the cells were stained with DAPI. The 96 well plate was placed on a motorized stage and both phase contrast and fluorescent images (FIG. 7a and FIG. 7b) were acquired using a Nikon TE-2000s camera controlled by the NIS Elements imaging software. After obtaining all images from the plate, a threshold from the phase contrast image (FIG. 7a) in the first well (without inhibitory compound) of the 96-well plate was adjusted to create a binary zone (FIG. 7c) between the pit area and area outside of the pit. The defined binary zone was applied to the DAPI image (FIG. 7b) to create two zones (zone 1 & zone 2) (FIG. 7d). Invaded cells in zone 2 (FIG. 7d) were then automatically counted (FIG. 7e). The lighter shade of particles in the right hand portion of FIG. 7e depict invaded cells. Based on the given threshold, the invaded cells in remaining wells were determined.

### Example 2

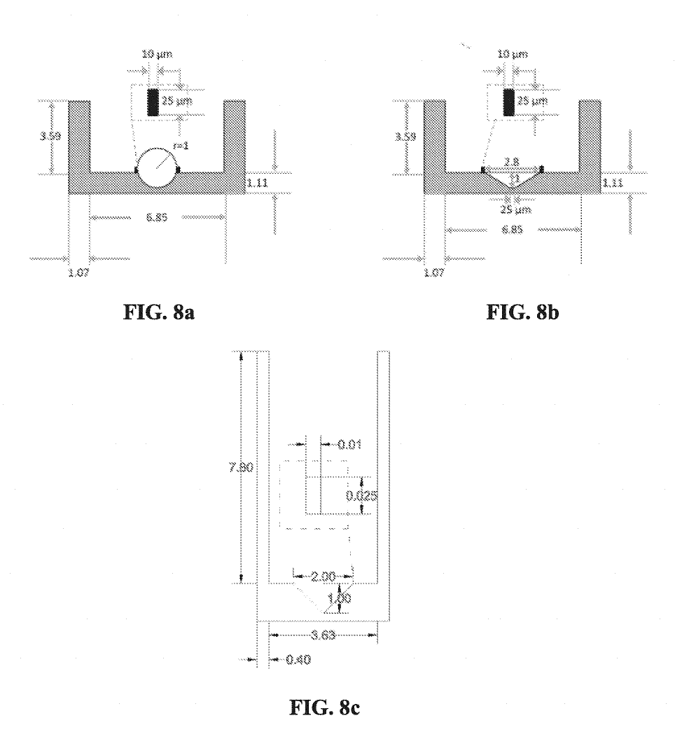

FIGS. 8a-8c are cross sectional view of two embodiments of wells, pits and pit barriers. Although dimensions are included in these figures, these dimensions are for illustrative purposes only, are in millimeters unless otherwise noted and can be modified larger or smaller by 1%, 10%, 50%, 100% or more. In these embodiments the volume of the well itself is about 120 μL, and the volume of the pit is about 2.1 μL.

In FIG. 8b the “V” shaped well includes a substantially flat bottom portion measuring about 25 μm in length, which is at a depth of about 1 mm below the lower surface of the well.

In FIG. 8c, the “V” shaped well includes converging straight lines that meet at 1 mm below the lower surface of the well.

### Example 3

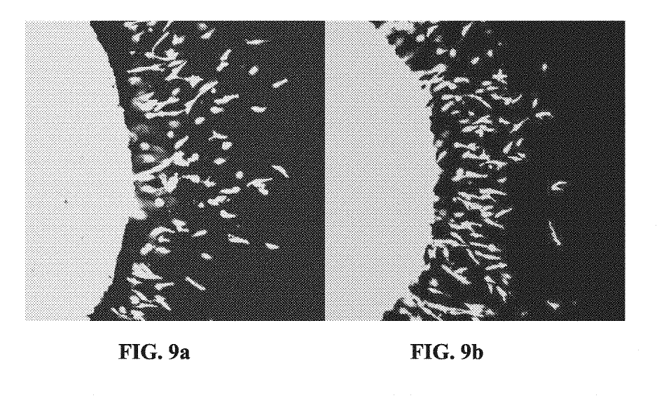

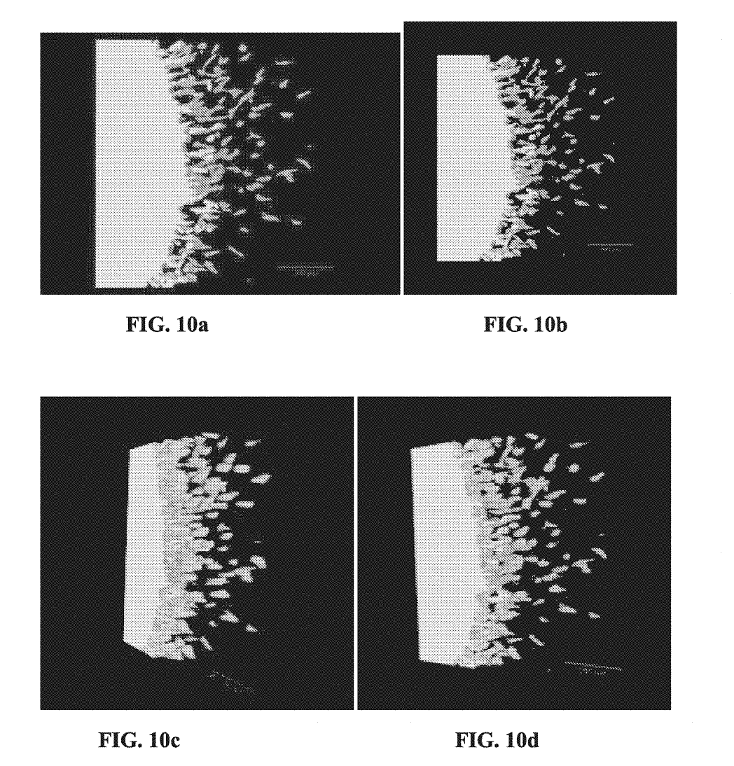

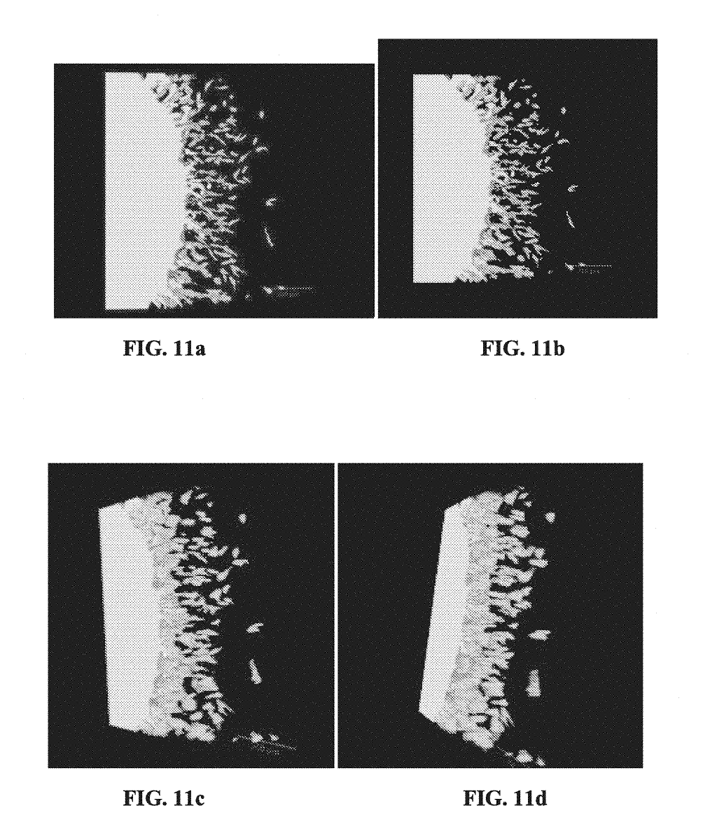

FIGS. 9a and 9b, 10a-10d and 11a-11d are fluorescence microscopy images of invasion zones showing Human-derived HT1080 GFP+ fibrosarcoma cells exiting the cell-collagen hemisphere (reference number 10 of FIG. 1b) and invading the extracellular matrix (reference number 14 of FIG. 1c) composed of type I collagen. In the three dimensional high-throughput invasion assay shown in FIGS. 9a and 9b, 10a-10d and 11a-11d, human-derived HT1080 fibrosarcoma cells engineered to stably express GFP were mixed with an equal volume of neutralized type I collagen on ice (1.5 mg/mL final concentration of type I collagen). The cell-collagen mixture (1 μL of 1×10⁷ cell/mL) was then dotted onto a 96-well plate. The well in FIGS. 9a and 10a-10d is of a structure similar to that shown in FIG. 8a, with the well in FIGS. 9b and 11a-11d being a structure similar to that shown in FIG. 8b.

After solidification, the cell-collagen hemispheres were covered with a layer of 1.5 mg/mL neutralized type I collagen. After solidification of cover collagen at 37° C., complete media was added and invasion assays were incubated for 18 hours overnight (37° C., 5% CO₂) and then fixed the next day with 4% paraformaldehyde and counterstained with DAPI nuclear dye before imaging. 10× microscopy images (FIGS. 9a, 9b) were captured for each quadrant/invasive edge (top, bottom, left, right) of the cell-collagen hemispheres on a Zeiss Confocal Microscope.

Three-dimensional reconstruction of the confocal images (z-stacks) taken of HT1080 GFP+ fibrosarcoma cells depicted in FIG. 9a are shown in FIGS. 10a-10d. FIG. 10a is a three dimensional rotation image of FIG. 9a, with FIGS. 10b-10d being three dimensional reconstructions of different angles of FIG. 9a.

Three-dimensional reconstruction of the confocal images (z-stacks) taken of HT1080 GFP+ fibrosarcoma cells depicted in FIG. 9b are shown in FIGS. 11a-11d. FIG. 11a is a three dimensional rotation image of FIG. 9b, with FIGS. 11b-11d being three dimensional reconstructions of different angles of FIG. 9b.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A plate comprising:
one or more wells, wherein each of the one or more wells comprises a bottom surface;
a pit formed in the bottom surface; and
a pit barrier formed around a periphery of the pit.

2. The plate of claim 1, wherein the one or more wells are arranged in a two-dimensional linear array pattern.

3. The plate of claim 1, wherein a height of the pit barrier is about 0.001 mm to about 1 mm.

4. The plate of claim 1, wherein a height of the pit barrier is about 0.01 mm to about 1 mm.

5. The plate of claim 1, wherein a height of the pit barrier is about 0.1 mm to about 1 mm.

6. The plate of claim 1, wherein a width of the pit barrier is about 0.001 mm to about 1 mm.

7. The plate of claim 1, wherein a width of the pit barrier is about 0.01 mm to about 1 mm.

8. The plate of claim 1, wherein a height of the pit barrier is about 0.1 mm to about 1 mm.

9. The plate of claim 1, wherein the bottom surface, the pit barrier and the pit form a continuous surface with each of the one or more wells.

10. The plate of claim 1, wherein the shape of the pit is selected from the group consisting of a circular cross-section and a V-shaped cross section and a combination of a flat well bottom and “V” shaped side walls.

11. The plate of claim 1, wherein the plate comprises 1 well, 2 wells, 4 wells, 6 wells, 12 wells, 24 wells, 48 wells, 96 wells, 384 wells or 1536 wells.

12. The plate of claim 1, wherein the plate comprises 6 wells, 12 wells, 24 wells, 48 wells, 96 wells or 384 wells.

13. The plate of claim 1, wherein the plate comprises 96 wells or 384 wells.

14. A method of producing a loaded plate, the method comprising:
adding a cell-matrix mixture to a pit of a plate, the plate comprising:
one or more wells, wherein each of the one or more wells comprises a bottom surface;
the pit formed in the bottom surface; and
a pit barrier formed around a periphery of the pit;
adding a matrix that substantially covers the cell-matrix mixture in the one or more wells.

15. The method of claim 14, wherein the shape of the pit is selected from the group consisting of a circular cross-section and a V-shaped cross section and a combination of a flat well bottom and “V” shaped side walls.

16. The method of claim 14, wherein the plate comprises 1 well, 2 wells, 4 wells, 6 wells, 12 wells, 24 wells, 48 wells, 96 wells, 384 wells or 1536 wells.

17. The method of claim 14, wherein the plate comprises 6 wells, 12 wells, 24 wells, 48 wells, 96 wells or 384 wells.

18. The method of claim 14, wherein the plate comprises 96 wells or 384 wells.

19. A cell culture plate comprising the plate according to claim 1.
